(19) United States
(12) Patent Application Publication
(10) Pub. No.: US 2017/0020487 A1
(43) Pub. Date: Jan. 26, 2017

(54) METHOD AND APPARATUS FOR COMPOUNDING ULTRASONIC IMAGES

(71) Applicant: ALPINION MEDICAL SYSTEMS CO., LTD., Hwaseong-si, Gyeonggi-do (KR)

(72) Inventors: Sun-yeob CHANG, Seoul (KR); Keonho SON, Seongnam-si (KR); Jonghoon KIM, Yongin-si (KR); Hyunchul CHO, Ansan-si (KR)

(21) Appl. No.: 15/039,507

(22) PCT Filed: Nov. 29, 2013
(86) PCT No.: PCT/KR2013/010973

§ 371 Date: May 26, 2016

(30) Foreign Application Priority Data
Nov. 29, 2013 (KR) 10-2013-0146923

(51) Int. Cl. A61B 8/08; G01S 7/52; G01S 15/89; A61B 8/00

(52) U.S. Cl. CPC A61B 8/5253; A61B 8/4483; G01S 7/52085; G01S 15/8995; G06T 5/50

(57) ABSTRACT

A method and an apparatus for compounding ultrasound images are disclosed. A method and an apparatus for compounding ultrasound images are provided to compound frames generated based on reflection signals generated upon receiving reflected transmissions of focused ultrasounds and unfocused ultrasounds to a subject so as to prevent a decreased frame rate as well as moving artifacts from affecting the ultrasound imaging due to movements of the subject.

### TECHNICAL FIELD

The present disclosure relates to a method and an apparatus for compounding ultrasound images.

### BACKGROUND

The statements in this section merely provide background information related to the present disclosure and do not necessarily constitute prior art.

The ultrasound system transmits an ultrasound to a subject by using a probe, and then receives a reflection signal reflected from the subject to generate an ultrasound image by converting the received reflection signal into an electric signal. The ultrasound system has noninvasive and nondestructive characteristics, and hence it is widely used in a medical field to acquire internal information of a body. The ultrasound system is importantly used in the medical field, since it is capable of providing the image of an internal tissue of the body without a surgical operation which physically incises the body to open view.

In general, the ultrasound system acquires data by focusing ultrasound scanline by scanline through a transmission focusing and generates one image frame by combining data of respective lines, in order to secure the quality of images.

In recent years, an image compounding technology has come to the fore to improve the quality of ultrasound images further. There are technologies for the ultrasound system to generate the images by spatially compounding a plurality of frames, utilize a frequency compound for compounding the images according to different frequencies, or generate the images through a dynamic receive beamforming process.

The spatial compound technology transmits the ultrasound beams or ultrasounds to different directions repeatedly, generates a plurality of frames by using the reception signal reflected from the subject, and then, acquires the final image by compounding these with each other to display. Accordingly, despite its improvement of the quality of images, the spatial compound technology requires a plurality of frames for generating one image frame to be displayed, which not only lowers the frame rate but also causes moving artifacts to occur when the subject is moved during the imaging process.

The frequency compound technology repeatedly transmits the ultrasounds having different frequencies, generates a plurality of frames by using the reception signal reflected from the subject, and then acquires the final image by compounding these with each other. And therefore, the frequency compound technology still suffers from the lowered frame rate and the moving artifacts similar to the spatial compound technology.

And therefore, there is a need for an ultrasound image technology capable of improving the quality of images while minimizing the deterioration of frame rate and the generation of moving artifact.

### DISCLOSURE

### Technical Problem

Some embodiments of the present disclosure provide an ultrasonic image compounding method and apparatus which combine frames generated based on the reflected signals received by sending a focused ultrasound and unfocused ultrasound to the object, without a deteriorated frame rate and subjecting to no influences by moving artifacts due to movements of the subject.

### SUMMARY

In accordance with some embodiments of the present disclosure, an ultrasound medical apparatus including a transducer, a beamformer and a compounding unit. The transducer is configured to transmit at least one focused ultrasound and at least one unfocused ultrasound to a subject, and to receive one or more first reflection signals corresponding to the focused ultrasound and one or more second reflection signals corresponding to the unfocused ultrasound. The beamformer is configured to generate focused frame data based on the first reflection signals, and to generate unfocused frame data based on the second reflection signals. And the compounding unit is configured to compound the focused frame data and the unfocused frame data into a single frame to generate final frame data.

In accordance with some embodiments of the present disclosure, a method, performed by an ultrasound medical apparatus, for compounding images includes

performing a focused ultrasound transceiving including
transmitting at least one focused ultrasound to a subject, and
receiving, from the subject, one or more first reflection signals corresponding to the focused ultrasound;

generating focused frame data based on the first reflection signals;

performing an unfocused ultrasound transceiving including
transmitting at least one unfocused ultrasound to the subject, and
receiving, from the subject, one or more second reflection signals corresponding to the unfocused ultrasound;

generating unfocused frame data to the subject based on the second reflection signals, and

compounding the focused frame data and the unfocused frame data into a single frame data to generate a final frame data.

### Advantageous Effects

In the focused ultrasound imaging, the ultrasounds are focused scanline by scanline to generate data, and a frame is generated by combining the data of the scanlines. Whereas, in the unfocused ultrasound imaging, a frame is generated by a singular ultrasound transmission. According to some embodiments of the present disclosure, the focused ultrasound-generated frame is compounded with the unfocused ultrasound-based frame, which excels in minimizing the deterioration of frame rate while improving the image quality beyond the focused ultrasound imaging method, and keeps the moving artifacts minimized by reducing the time required for the data acquisition and processing.

Moreover, the frequency compound technology or the spatial compound technology according to some embodiments of the present disclosure provides a superior improvement of the frame rate while minimizing the deterioration of image quality with reduced moving artifacts in comparison with the frequency compound and the spatial compound methods using only the focused frame data. Further, the entire processing time to acquire, generate and compound the images can be reduced.

### BRIEF DESCRIPTION OF DRAWINGS

FIG. 1 is a schematic block diagram of an ultrasound medical apparatus according to at least one embodiment of the present disclosure.

FIG. 2 is a diagram of a generation of a final frame through a frame compounding according to at least one embodiment of the present disclosure.

FIG. 3A is a diagram of a process of generating a focused frame according to at least one embodiment of the present disclosure.

FIG. 3B is a diagram of a process of generating an unfocused frame according to at least one embodiment of the present disclosure.

FIG. 4 is a flowchart of a method for compounding ultrasound images according to at least one embodiment of the present disclosure.

FIG. 5 is an exemplary diagram of a frame compounding cycle according to at least one embodiment of the present disclosure.

REFERENCE NUMERALS

| | |
|---|---|
| 100: ultrasound medical apparatus | 110: transducer |
| 120: front end | 122: transceiving unit |
| 124: analog-to-digital converter | 130: host |
| 132: beamformer | 134: compounding unit |
| 136: signal processing unit | 138: scan converting unit |

### DETAILED DESCRIPTION

Hereinafter, at least one embodiment of the present disclosure will be described in detail with reference to the accompanying drawings.

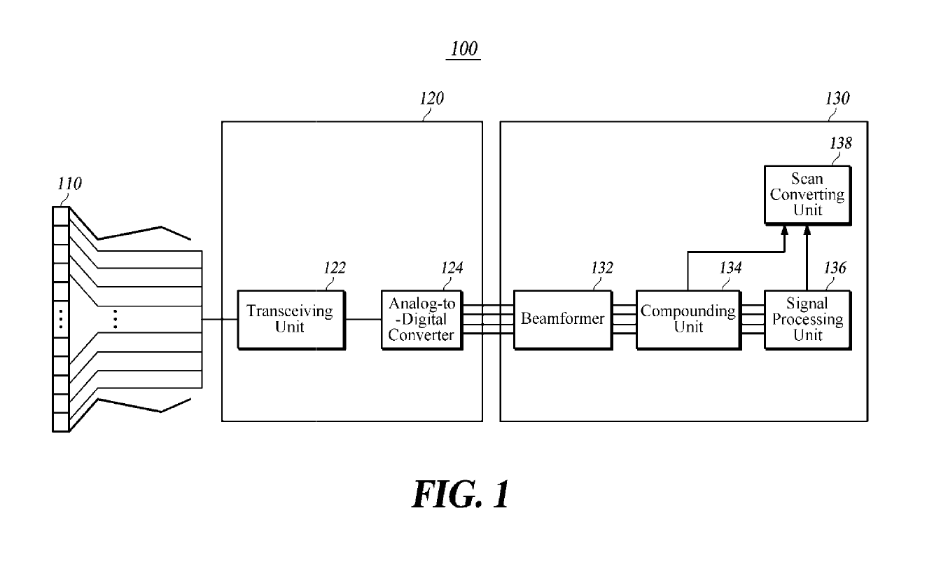

FIG. 1 is a schematic block diagram of an ultrasound medical apparatus 100 according to at least one embodiment of the present disclosure.

The ultrasound medical apparatus 100 performs a software-based beamforming, and includes a transducer 110, a front end 120 and a host 130. The ultrasound medical apparatus 100 in some embodiments of the present disclosure is not necessarily limited to this configuration.

The front end 120 may include a transceiving unit 122 and an analog-to-digital converter 124. The host 130 may include a beamformer 132, a compounding unit 134, a signal processing unit 136 and a scan converting unit 138. In this configuration, the host 130 performs a software-based parallel processing for the purpose of a fast imaging process, and, in terms of architecture, can perform the parallel processing in a plurality (for example, several thousands) of processors at the same time with a multi-core CPU (Central Processing Unit) and a GPU (Graphic Processing Unit).

For the purpose of the software-based high-speed imaging processing, the front end 120 and the host 130 may be connected through a full parallel path, e.g., a PCI (Peripheral Component Interconnect Express) interface.

The ultrasound medical apparatus 100 according to the embodiments of the present disclosure performs the fast imaging process on the basis of software, which facilitates the compounding process of the ultrasound image thanks to the connection structure of the full parallel path between the front end 120 and the host 130. When a user desires to view an image with high quality according to types of subjects or the purpose of diagnosis, the ultrasound medical apparatus 100 can compound unfocused frame data based on focused image data to provide a high quality ultrasound image in a short time.

The transducer 110 converts an electrical analog signal into an ultrasound, transmits the ultrasound to a subject, receives a signal reflected at the subject (hereinafter, a “reflected signal”), and converts the reflected signal into an electrical analog signal. The transducer 110 may be implemented with a transducer array, transmits the ultrasound to the subject by using transducer elements in the transducer array, and receives a reflected signal from the subject. The transducer 110 transmits the reflected signal inputted from the subject to the front end 120 which then transfers the received reflected signal to the beamformer 132.

After transmitting the focused ultrasound to the subject, the transducer 110 according to some embodiments receives the first reflected signal corresponding to the focused ultrasound from the subject. Under the control of the transceiving unit 122, the transducer 110 focuses and transmits the ultrasound at every scanline, and receives the first reflected signal for each scanline. After transmitting the unfocused ultrasound to the subject at least one time, the transducer 110 receives the second reflected signal corresponding to the unfocused ultrasound from the subject. In some embodiments, the unfocused ultrasound includes a beam of at least one of a plane wave and a broad beam. The second reflected signal may be subjected to a software-based image high-speed processing.

The transducer 110 transmits the focused ultrasound to the subject during a first transceiving period according to the control of the transceiving unit 122, and transmits the unfocused ultrasound at least one time during a second transceiving period. The first transceiving period and the second transceiving period have transceiving timings different from each other. The operation of the transducer 110 under the control of the transceiving unit 122 starts with the first transceiving period in which the transducer 110 transmits the focused ultrasound along the scanline to the subject. During the second transceiving period, the transducer 110 transmits the unfocused ultrasound at least once to the subject by using all scanlines.

The unfocused ultrasounds transmitted to the subject by the transducer 110 may have frequencies different from those of the focused ultrasounds, and the frequencies of the unfocused ultrasounds may be different from each other. Further, the transducer 110 may transmit the unfocused ultrasounds with a plurality of different transmission angles to the subject. In other words, the transducer 110 may transmit the unfocused ultrasounds having a predetermined phase difference to the subject.

The following will describe the components of the front end 120.

The transceiving unit 122 applies a voltage pulse to the transducer 110 to output the focused ultrasound or the unfocused ultrasound from each transducer element of the transducer 110. The transceiving unit 122 serves as a transmission/reception switch for switching the transducer 110 to perform a transmission and reception alternately.

The transceiving unit 122 according to some embodiments controls the transducer 110 to transmit the focused ultrasound to the subject during the first transceiving period. The transceiving unit 122 controls the transducer 110 to transmit the unfocused ultrasound at least once to the subject during the second transceiving period. The transceiving unit 122 operates to interleave the first transceiving period with the second transceiving period. After the analog-to-digital converter 124 converts the analog reflection signal received from the transceiving unit 122 into the digital signal, and then transmits the latter to the host 130.

The following will describe the components of the host 130.

The beamformer 132 delays electrical signals appropriate to the transducer 110 to convert the electrical signals into an electrical signal matched to each of the transducer elements. In addition, the beamformer 132 delays or sums electrical signals respectively converted by the transducer elements for calculating the delayed or summed electrical signals as frame data or scanline data of the corresponding transducer elements. The beamformer 132 includes a transmit beamformer, a receive beamformer and a beamforming unit. In some embodiments, the beamformer 132 is connected to the analog-to-digital converter 124 and the signal processing unit 136 via a full parallel path, in order to perform the software-based high-speed image processing.

The beamformer 132 according to some embodiments generates the focused frame data by using the first reflection signals obtained scanline by scanline, and generates the unfocused frame data based on the second reflection signal. As for the process of generating the frame data by the beamformer 132, the beamformer 132 generates the focused frame data with the first reflection signals equivalent to the number of scanlines of the transducer 110. The beamformer 132 also generates the unfocused frame data from the second reflection signal. Meanwhile, when the unfocused ultrasound is transmitted multiple times, a plurality of unfocused frame data may be generated by using the respective second reflection signals corresponding to the multiple transmissions; and the unfocused frame data may be further compounded into a single unfocused frame data.

The beamformer 132 generates at least one frame as the focused frame data based on the first reflection signal. For example, when the transducer 110 transmits the focused ultrasound to the subject, the beamformer 132 may receive the first reflection signal corresponding to the focused ultrasound from the subject, and then generate the focused frame data. It is preferable that the beamformer 132 receives the reflection signals for all the scanlines of the transducer 110 to generate a single frame (focused frame), but the disclosure is not limited thereto. For example, the beamformer 132 may repeat to receive the reflection signals for all scanlines to generate a plurality of frames and compound them into a single frame (focused frame).

The beamformer 132 generate at least two frames as the unfocused frame data based on the second reflection signal. For example, when the transducer 110 transmits the unfocused ultrasounds having a plurality of different transmission angles to the subject, the beamformer 132 may receive the second reflection signals corresponding to the unfocused ultrasounds having a plurality of different transmission angles from the subject, and then generate a singular unfocused frame data by spatially compounding the frames with the respective transmission angles.

In the process performed by the beamformer 132 for generating the frames with the second reflection signal corresponding to the unfocused ultrasound, the beamformer 132 spatially compounds the signals at the completion of a receive beamforming process, or perform a frequency compounding on the signals prior to the receive beamforming process. The beamformer 132 stores the first reflection signal in the storing unit at the completion of the reception beamforming, or stores the second reflection signal in the storing unit at a time prior to the receive beamforming process. Here, the reflection signals stored in the storing unit prior to the receive beamforming process refer to raw data.

The compounding unit 134 according to some embodiments compounds the focused frame data and the unfocused frame data into one frame to generate the final frame data. The compounding unit 134 applies a predetermined weight to each of the focused frame data and the unfocused frame data to generate the final single compounded frame data. For example, the compounding unit 134 can generate the unfocused frame data based on the focused frame data, and therefore it is preferable that a high weight is applied to the focused frame data and a low weight is applied to the unfocused frame data, but the disclosure is not limited thereto. In other words, with different weights applied to each of the focused frame data and the unfocused frame data, the compounding unit 134 may generate the final single compounded frame data.

The signal processing unit 136 converts the reflection signals of the reception scanline focused by the beamformer 132 into the baseband signals, and obtains data of the frame or at least one scanline by detecting an envelope using a quadrature demodulator. The signal processing unit 136 processes the data generated by the beamformer 132 into a digital signal. Further, the signal processing unit 136 may receive and post-process the final frame data from the compounding unit 134.

The scan converting unit 138 aligns the scan direction of data obtained from the beamformer 132 with the pixel direction of a display unit (e.g., a monitor), and maps the corresponding data to the pixel positions on the display unit. The scan converting unit 138 converts ultrasound image data into a data format used in the display unit having a predetermined scanline display format.

In some embodiments, the ultrasound medical apparatus 100 may further include a user input unit which receives instructions from an operation or an input of a user. In some embodiments, the user instructions include a setting instruction for controlling the ultrasound medical apparatus 100 and the like. Further, the ultrasound medical apparatus 100 may include a storing unit for storing a reflection signal (a signal before performing the receive beamforming) that passed through the analog-to-digital converter 124 or a reflection signal (a signal at the time of finishing a reception beamforming) after the receive beamforming process.

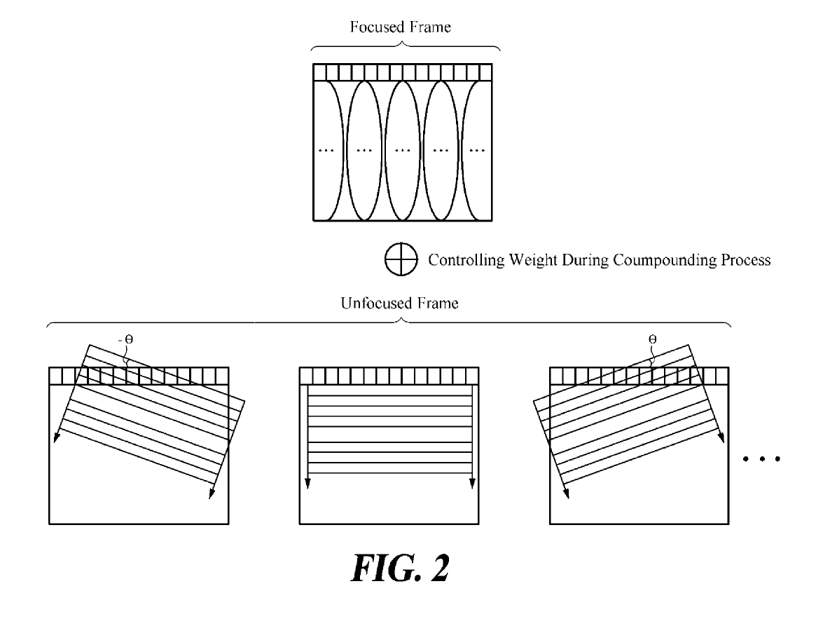

FIG. 2 is a diagram of a generation of a final focused frame through a frame compounding according to at least one embodiment of the present disclosure.

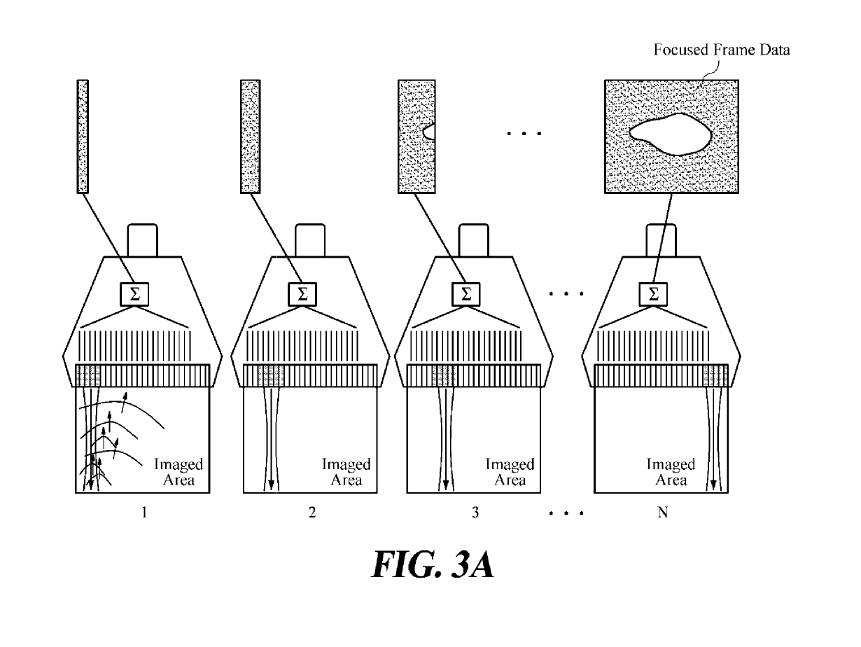

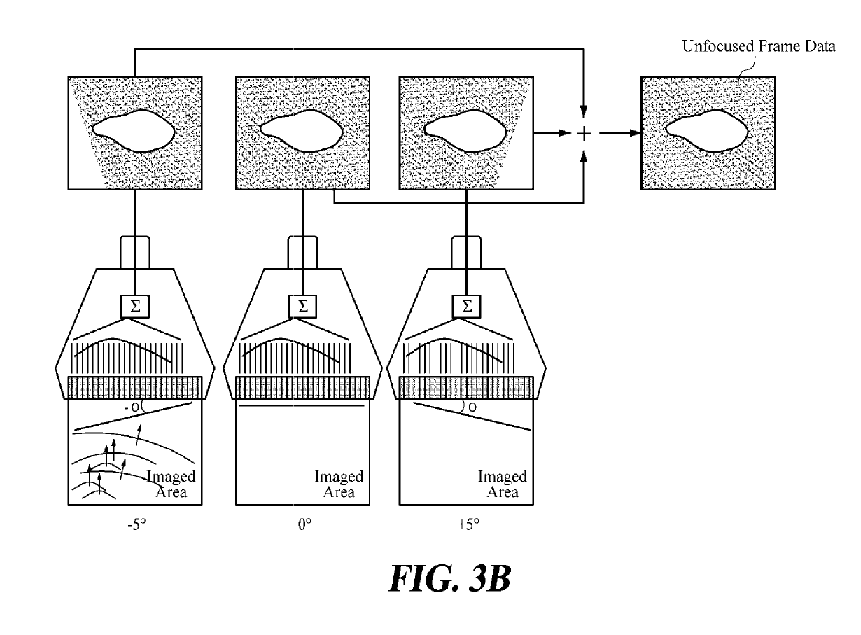

As shown in FIG. 2, after transmitting the focused ultrasound to the subject, the ultrasound medical apparatus 100 receives the first reflection signal corresponding to the focused ultrasound from the subject, and generates the focused frame data based on the first reflection signal. The process to generate the focused frame data by the ultrasound medical apparatus 100 will be explained in detail through FIG. 3A hereinbelow.

After the ultrasound medical apparatus 100 transmits the unfocused ultrasound to the subject, it receives the second reflection signal corresponding to the unfocused ultrasound from the subject, and it generates the unfocused frame data based on the second reflection signal. The process to generate the unfocused frame data by the ultrasound medical apparatus 100 will be explained in detail through FIG. 3B hereinbelow.

Referring to FIG. 2, a method performed by the ultrasound medical apparatus 100 for varying the transmission angles of the unfocused ultrasounds will be described. The ultrasound medical apparatus 100 may transmit the unfocused ultrasounds having phase difference ‘−θ’ from adjacent transducer elements to the subject. The ‘θ’ shown in FIG. 2 conceptually represents the phase difference between the adjacent transducers as ‘θ’ rather than a physically moving angle of the transducer. Thereafter, the ultrasound medical apparatus 100 generates the frame based on the reflection signals corresponding to the adjacent unfocused ultrasounds having phase difference ‘−θ’.

The ultrasound medical apparatus 100 transmits the unfocused ultrasounds with no phase difference from the adjacent transducer elements to the subject, and generates the frame based on the reflection signals corresponding to the adjacent unfocused ultrasounds with no phase difference. After transmitting the adjacent unfocused ultrasounds having phase difference ‘θ’ to the subject, the ultrasound medical apparatus 100 generates the frame based on the reflection signals corresponding to the adjacent unfocused ultrasounds having phase difference ‘θ’. The ultrasound medical apparatus 100 generates a single unfocused frame data by using the frame corresponding to the adjacent unfocused ultrasounds having phase difference ‘−θ’, the frame corresponding to the adjacent unfocused ultrasounds with no phase difference and the frames corresponding to the adjacent unfocused ultrasounds having phase difference ‘θ’.

Thereafter, the ultrasound medical apparatus 100 generates the final frame data by compounding the focused frame data and the unfocused frame data.

FIG. 3A is a diagram of a process of generating a focused frame according to at least one embodiment of the present disclosure.

As shown in FIG. 3A, the generation of the focused frame data by the ultrasound medical apparatus 100 includes generating a partial image of the frame by using one ultrasound beam for each scanline, and subsequently generating one frame by using these partial images.

At first, the ultrasound medical apparatus 100 transmits the focused ultrasound to the subject along a predetermined scanline and then receives the first reflection signal from the subject. The ultrasound medical apparatus 100 generates the focused frame data based on the first reflection signal for each scanline. At this time, the generated focused frame data may be outputted separately through the display unit provided in the ultrasound medical apparatus 100 independently of the compounding of the unfocused frame data. For example, as shown in FIG. 3A, when there are the first through the N-th scanlines, the ultrasound medical apparatus 100 transmits the focused ultrasound along the first scanline and then receives the reflection signal to perform the imaging process, and repeats the same process up to the N-th scanline, in order to yield the focused frame data.

FIG. 3B is a diagram of a process of generating an unfocused frame according to at least one embodiment of the present disclosure.

As shown in FIG. 3B, the frame processed by the ultrasound medical apparatus 100 by generating the unfocused ultrasound is obtained faster than conventional imaging methods because all the transducer elements are involved at a time to produce the unfocused frame data. For example, the ultrasound medical apparatus 100 transmits the unfocused ultrasound to the subject, and generates the unfocused framed data based on the second reflection signal corresponding to the unfocused ultrasound. At this time, the generated unfocused frame data may be outputted through the display unit provided in the ultrasound medical apparatus 100 independently of compounding the focused frame data.

Referring to FIG. 3B, a method performed by the ultrasound medical apparatus 100, for generating an image by differentiating the transmission angles of the unfocused ultrasounds. When generating the unfocused frame data based on the second reflection signal, the ultrasound medical apparatus 100 may perform a software-based parallel processing for the purpose of a fast imaging process. In addition, when transmitting the unfocused ultrasound to the subject, the ultrasound medical apparatus 100 may control to have a plurality of different transmission phase differences (for example, −θ, θ).

As shown in FIG. 3B, the ultrasound medical apparatus 100 transmits the unfocused ultrasounds having phase difference of ‘−5°’ from the adjacent transducer elements, and generates the frame based on the reflection signal corresponding to the unfocused ultrasound having phase difference of ‘−5°’ from the adjacent transducer elements.

Moreover, the ultrasound medical apparatus 100 transmits the unfocused ultrasounds with no phase difference from the adjacent transducer elements, and generates the frame based on the reflection signals corresponding to the adjacent unfocused ultrasounds with no phase difference. The ultrasound medical apparatus 100 transmits the adjacent unfocused ultrasounds having phase difference of ‘+5°’ to the subject, and generates the frame based on the reflection signals corresponding to the unfocused ultrasounds having phase difference of ‘+5°’ with respect to a level surface of the transducer. Thereafter, The ultrasound medical apparatus 100 generates a single unfocused frame data by using the frame corresponding to the adjacent unfocused ultrasounds having phase difference ‘−θ’, the frame corresponding to the adjacent unfocused ultrasounds with no phase difference and the frames corresponding to the adjacent unfocused ultrasounds having phase difference ‘θ’.

Meanwhile, the ultrasound medical apparatus 100 transmits the unfocused ultrasounds to the subject, to generate the frame based on the reflection signals corresponding to the unfocused ultrasounds, and then, transmits the unfocused ultrasounds having different frequencies again to the subject, to generate the frame based on the reflection signals corresponding to the unfocused ultrasounds having the different frequencies. Thereafter, the ultrasound medical apparatus 100 may generate unfocused frame data by based on the frame corresponding to the unfocused ultrasounds and the frames corresponding to the different unfocused ultrasounds.

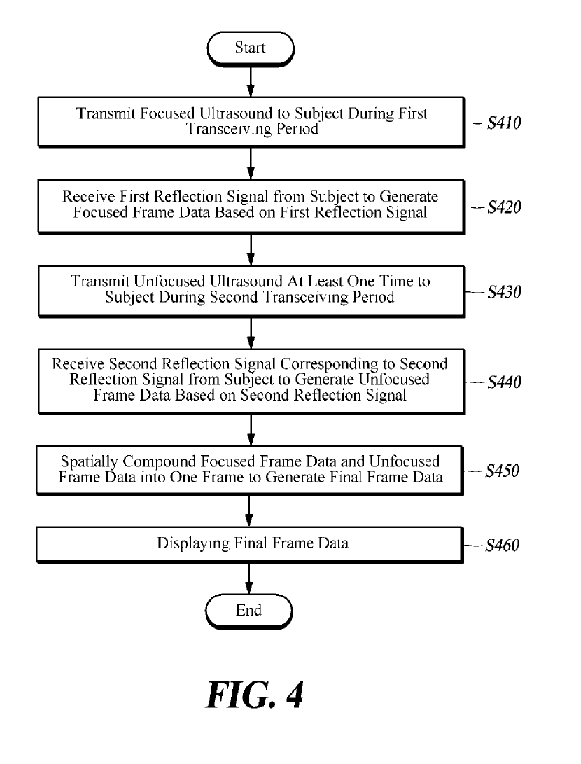

FIG. 4 is a flowchart of a method for compounding ultrasound images according to at least one embodiment of the present disclosure.

An ultrasound medical apparatus 100 transmits a focused ultrasound to a subject during at least one first transceiving period (Step S410). In Step S410, the first transceiving period refers to the interval that lasts until the focused ultrasound transmission completes along the scanline of the transducer 110 of the ultrasound medical apparatus 100. For example, provided there are 128 scanlines from the transducer 110 of the ultrasound medical apparatus 100, the first transceiving period lasts until the focused ultrasound transmission completes along the scanlines of 128 elements.

The ultrasound medical apparatus 100 receives one or more first reflection signals from the subject, and generates focused frame data based on the first reflection signals (Step S420). In Step S420, the ultrasound medical apparatus 100 generates the focused frame data with the first reflection signals by the number of scanlines (e.g., 128). The ultrasound medical apparatus 100 may generate at least one frame with the focused frame data based on the first reflection signals.

The ultrasound medical apparatus 100 transmits the unfocused ultrasound at least one time to the subject during at least one second transceiving period (Step S430). In Step S430, the second transceiving period is different from the first transceiving period, and is shorter than the first transceiving period. Since the ultrasound medical apparatus 100 uses all scanlines of the transducer 110 at one time for the purpose of transmitting the unfocused ultrasound, it takes the shorter second transceiving period than the first transceiving period for using all scanlines of the transducer 110 to transmit the unfocused ultrasound to the subject.

Further, the ultrasound medical apparatus 100 transmits, to the subject, the unfocused ultrasound (having different frequencies between themselves) having frequencies different from those of the focused ultrasounds, and may transmit, to the subject, the unfocused ultrasounds having a plurality of different transmission angles. In some embodiments, the unfocused ultrasound includes a beam of at least one of a plane wave and a broad beam.

The ultrasound medical apparatus 100 receives one or more second reflection signals corresponding to the second reflection signals from the subject, and generates unfocused frame data based on the second reflection signals (Step S440). In Step S440, the ultrasound medical apparatus 100 generates the unfocused frame data with a predetermined number of the second reflection signals. For example, given that the predetermined number is ‘2’, the ultrasound medical apparatus 100 generates the unfocused frame data by using 2 sequences of the second reflection signals.

The ultrasound medical apparatus 100 generates at least two frames as the unfocused frame data based on the second reflection signals. For example, when there are at least two frames generated by using the second reflection signal corresponding to the unfocused ultrasound, the ultrasound medical apparatus 100 may spatially compound the signals at the time of completing the receive beamforming, or may generate the unfocused frame data by performing a frequency compounding on the signals prior to performing the receive beamforming.

The ultrasound medical apparatus 100 spatially compounds the focused frame data and the unfocused frame data into one frame to generate the final frame data (Step S450). In Step S450, the ultrasound medical apparatus 100 may generate the final frame data which is compounded into the single frame, by applying a predetermined weight to each of the focused frame data and the unfocused frame data.

After Step S450, the ultrasound medical apparatus 100 may store the first reflection signals at the time of completing the receive beamforming, or may store the second reflection signals before performing the receive beamforming. The ultrasound medical apparatus 100 renders the final frame data to be displayed through a display unit (S460).

Although Steps S410 to S460 are described to be sequentially performed in the example shown in FIG. 4, it merely instantiates a technical idea of some embodiments of the present disclosure. Therefore, a person having ordinary skill in the pertinent art could appreciate that various modifications, additions, and substitutions are possible by changing the sequences described in FIG. 4 or by executing two or more steps in parallel, without departing from the gist and the nature of the embodiments of the present disclosure, and hence FIG. 4 is not limited to the illustrated chronological sequence.

The ultrasound image compound method according to the embodiment shown in FIG. 4 may be implemented as a computer program, and may be recorded on a computer-readable medium. The computer-readable recording medium on which the ultrasound image compounding method according to the embodiment is recordable includes any type of recording device on which data that can be read by a computer system are recordable.

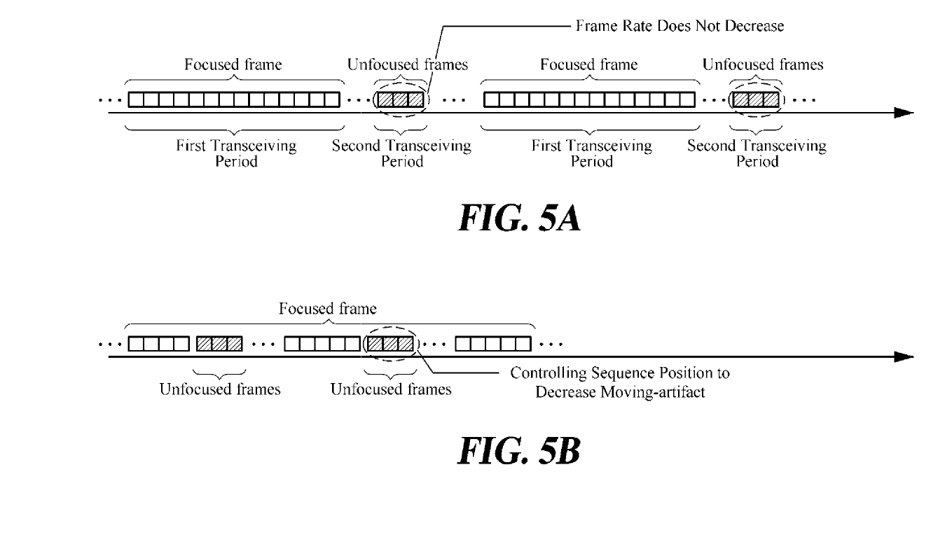

FIG. 5A and FIG. 5B are exemplary diagrams of a frame compounding cycle according to at least one embodiment of the present disclosure.

Referring to FIG. 5A for example, the ultrasound medical apparatus 100 transmits the focused ultrasounds to the subject along the scanlines to receive the first reflection signals from the subject. The ultrasound medical apparatus 100 generates the focused frame data based on the first reflection signal for each scanline. And then, the ultrasound medical apparatus 100 transmits the unfocused ultrasounds to the subject by using all scanlines at one time to receive the second reflection signals from the subject. The ultrasound medical apparatus 100 generates the unfocused frame data based on the second reflection signals. At this time, the unfocused frame data may be composed of approximately 1 to 3 sequences. For example, transmitting the unfocused ultrasounds having a plurality of different transmission angles to the subject, the ultrasound medical apparatus 100 may generate the frame for each of the transmission angles.

When the focused frame data and the unfocused frame data are generated, as shown in FIG. 5A, the ultrasound medical apparatus 100 operates the transducer 110 to transmit the focused ultrasounds to the subject during the first transceiving period, and to transmit the unfocused ultrasound at least one time during the second transceiving period.

Further, as shown in FIG. 5A, when approximately 1 to 3 pieces of unfocused frame data are generated and compounded with the focused frame data, the frame rate of the compounded final frame data does not decrease. In other words, since the unfocused frame data including approximately 1 to 3 sequences does not require as large data acquisition time as with the focused frame data, the final frame data obtained by compounding the unfocused frame data based on the focused frame data does not suffer from a decreased frame rate.

The first transceiving period, shown in FIG. 5A, is the time period required for acquiring the data for at least one frame by using the focused ultrasound. The second transceiving period, shown in FIG. 5A, is the time period required for acquiring the data for at least one frame by using the unfocused ultrasound. Each of the second transceiving periods, shown in FIG. 5A, may exist between the first transceiving periods (the frame data acquisition periods).

Referring to FIG. 5B, the ultrasound medical apparatus 100 may operate to interleave the unfocused transmission sequences with the focused transmission periods. The ultrasound medical apparatus 100 may operate to transmit the focused ultrasounds to the subject along the scanline, transmit the unfocused ultrasounds to the subject by using all scanlines at one time between every two adjacent focused transmission periods and then receive the first reflection signals from the subject; and then insert unfocused transmission sequences which receive the second reflection signals corresponding to the unfocused ultrasounds. When the unfocused transmission sequences are inserted between every two adjacent focused transmission periods, as shown in FIG. 5B, the ultrasound medical apparatus 100 according to some embodiments can supply the ultrasound image with minimized effect of the moving artifacts due to movements of the subject.

The first transceiving period, shown in FIG. 5B, is the time period required for acquiring the data for at least one scanline by using the focused ultrasounds. The second transceiving period, shown in FIG. 5B, is the time period required for acquiring the data for at least one frame by using the unfocused ultrasounds. Each of the second transceiving period, shown in FIG. 5A, may exist between the first transceiving periods (the scanline data acquisition periods).

Although exemplary embodiments of the present disclosure have been described for illustrative purposes, those skilled in the art will appreciate that various modifications, additions and substitutions are possible, without departing from the idea and scope of the claimed disclosure. Specific terms used in this disclosure and drawings are used for illustrative purposes and not to be considered as limitations of the present disclosure. Therefore, exemplary embodiments of the present disclosure have been described for the sake of brevity and clarity. Accordingly, one of ordinary skill would understand the scope of the claimed disclosure is not to be limited by the explicitly described above embodiments but by the claims and equivalents thereof.

### CROSS-REFERENCE TO RELATED APPLICATION

This application claims priority under 35 U.S.C §119(a) of Patent Application No. 10-2013-0146923, filed on Nov. 29, 2013 in Korea, the entire content of which is incorporated herein by reference. In addition, this non-provisional application claims priority in countries, other than the U.S., with the same reason based on the Korean patent application, the entire content of which is hereby incorporated by reference.

1. An ultrasound medical apparatus, comprising:
a transducer configured to transmit at least one focused ultrasound and at least one unfocused ultrasound to a subject, and to receive one or more first reflection signals corresponding to the focused ultrasound and one or more second reflection signals corresponding to the unfocused ultrasound;
a beamformer configured to generate focused frame data based on the first reflection signals, and to generate unfocused frame data based on the second reflection signals; and
a compounding unit configured to compound the focused frame data and the unfocused frame data into a single frame to generate final frame data.

2. The ultrasound medical apparatus of claim 1, further comprising:
a transceiving unit configured to control the transducer to transmit the focused ultrasound to the subject during each of first transceiving periods, and to transmit the unfocused ultrasound to the subject at least one time during each of second transceiving periods.

3. The ultrasound medical apparatus of claim 2, wherein
the transducer is configured to perform a plurality of transmissions of the unfocused ultrasound to the subject during the second transceiving period, and
the beamformer is configured to compound the second reflected signals corresponding respectively to the plurality of transmissions to generate the unfocused frame data.

4. The ultrasound medical apparatus of claim 2, wherein
the focused ultrasound takes the first transceiving period to obtain data of at least one frame, and
each of the second transceiving periods resides in between two of the first transceiving periods.

5. The ultrasound medical apparatus of claim 2, wherein the focused ultrasound takes the first transceiving period to obtain data of at least one scanline, and each of the second transceiving periods resides in between two of the first transceiving periods.

6. The ultrasound medical apparatus of claim 1, wherein
the transducer is configured to transmit the unfocused ultrasound having a frequency different from that of the focused ultrasound to the subject, and
the beamformer is configured to generate at least one frame into the focused frame data based on the first reflection signal.

7. The ultrasound medical apparatus of claim 1, wherein
the transducer is configured to transmit unfocused ultrasounds having a plurality of different transmission angles or different frequencies to the subject, and
the beamformer is configured to generate at least two frames into the unfocused frame data based on the second reflection signals.

8. The ultrasound medical apparatus of claim 7, wherein the beamformer is configured to perform a spatial compounding of signals at the time of completing a receive beamforming or to perform a frequency compounding of signals at the time prior to performing the receive beamforming, when the at least two frames are generated into the unfocused frame data.

9. The ultrasound medical apparatus of claim 1, wherein the compounding unit is configured to generate the final frame data which is compounded into the single frame, by applying a predetermined weight to each of the focused frame data and the unfocused frame data.

10. The ultrasound medical apparatus of claim 1, wherein the beamformer is configured to store the first reflection signals at the time of completing a receive beamforming or to store the second reflection signals before performing the receive beamforming.

11. The ultrasound medical apparatus of claim 1, wherein the unfocused ultrasound comprises a beam of at least one of a plane wave and a broad beam.

12. A method, performed by an ultrasound medical apparatus, for compounding images, the method comprising:
performing a focused ultrasound transceiving comprising:
transmitting at least one focused ultrasound to a subject, and
receiving, from the subject, one or more first reflection signals corresponding to the focused ultrasound;
generating focused frame data based on the first reflection signals;
performing an unfocused ultrasound transceiving comprising:
transmitting at least one unfocused ultrasound to the subject, and
receiving, from the subject, one or more second reflection signals corresponding to the unfocused ultrasound;
generating unfocused frame data to the subject based on the second reflection signals; and
compounding the focused frame data and the unfocused frame data into a single frame data to generate a final frame data.

13. The method of claim 12, wherein
the focused ultrasound to the subject during each of first transceiving periods; and
the unfocused ultrasound is transmitted to the subject at least one time during each of second transceiving period.

14. The method of claim 13, wherein
the unfocused ultrasound is transmitted multiple times to the subject during the second transceiving period, and
the unfocused frame data is generated by compounding the second reflected signals corresponding respectively to multiple transmissions of the unfocused ultrasound.

15. The method of claim 13, wherein
the focused ultrasound takes the first transceiving period to obtain data of at least one frame, and
each of the second transceiving periods resides in between two of the first transceiving periods.
